(19) United States
(12) Patent Application Publication
(10) Pub. No.: US 2010/0191478 A1
(43) Pub. Date: Jul. 29, 2010

(54) METHOD FOR ESTIMATING THE LEVEL OF ETHANOL IN A FUEL

(75) Inventors: Pascal Emery, Palaiseau (FR); Marcos Navarro, Paris (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: RENAULT S.A.S., Boulogne-Billancourt (FR)

(21) Appl. No.: 12/597,594

(22) PCT Filed: Apr. 8, 2008
(86) PCT No.: PCT/FR08/50621

§ 371 Date: Feb. 26, 2010

(30) Foreign Application Priority Data
Apr. 25, 2007 (FR) 0703001

(51) Int. Cl. G01N 33/22; G06F 19/00

(52) U.S. Cl. 702/24

(57) ABSTRACT

A method for estimating a level of ethanol in a fuel injected into an internal combustion engine over a number (n) of cycles (n being an integer greater than or equal to 1). The method includes: determining start of combustion of an injected fuel for each of the cycles; calculating the average combustion start angle from combustion start angles recorded for all of the n cycles; determining the end of combustion of an injected fuel for each of the cycles; calculating the average combustion end angle from the combustion end angles recorded for all of the n cycles; calculating the combustion time between the combustion start time and the combustion end time; and deducting the level of ethanol from the combustion time on the basis of a pre-determined model or a list of combustion times and associated ethanol levels.

The invention falls within the field of internal combustion (gasoline) engines with direct or indirect fuel injection, and a number N of cylinders.

It relates more specifically to how to estimate the ethanol content of the fuel injected, this being discovered by determining the start and end of combustion of a fuel injected into an internal combustion engine over a number n of cycles (n being an integer greater than or equal to 1) of displacement of a piston in a cylinder.

In the face of ever-tightening emissions standards it is important for motor vehicle manufacturers to have good control over how the fuel is injected into their engines in order to limit pollutant emissions.

One of the important parameters in these injection systems is the type of fuel.

Particularly in the case of gasoline engines, the emergence of combustion systems based on “flex fuel” or fuels derived from a mixture of gasoline and of ethanol makes the basic setups defined during engine calibration considerably more complicated.

The concentration of ethanol in the fuel varies enormously according to the regulations in force in the various countries.

This concentration governs the self-ignition properties of the fuel, or in other words, the ignition delay.

This delay is important because it governs the timing of the combustion within the thermodynamic cycle, with respect to a given ignition advance setpoint. Poorly timed combustion will lead to an increase in pollutant emissions.

It also makes cold starting conditions more severe.

It is therefore extremely important to take the type of fuel present in the tank into consideration very accurately when setting up the engines.

At the present time, this concentration of ethanol in the fuel is taken into consideration during engine development. This is performed by using two reference fuels, for example a first one consisting of pure gasoline and a second containing 85% ethanol, the remaining 15% consisting of gasoline.

Two full optimizations are performed in order to define the engine settings optimally, for each fuel.

In operation, if the vehicle is run on a different fuel (that is to say a fuel with a different ethanol concentration), the engine will no longer operate at its optimum settings.

Engine management therefore uses the richness information to calculate an image of the alcohol content. Thereafter, interpolation is performed for each setting parameter between the two initial optimizations.

Solutions for estimating a quantity characteristic of the start of combustion has been the subject of French patent applications in the name of the same applicant, namely applications 05/07745 and 06/07041, the latter having not yet been published at the time of filing of the present application.

It is an object of the present invention to propose a method for estimating the ethanol content of the injected fuel, discovered from determining the start and end of combustion of a fuel injected into an internal combustion engine, over a number n of cycles (n being an integer greater than or equal to 1) of displacement of a piston in a cylinder.

To achieve this objective, the invention proposes a method for estimating the ethanol content of a fuel injected into an internal combustion engine, over a number n of cycles (n being an integer greater than or equal to 1) of displacement of a piston in a cylinder, characterized in that it comprises the following steps:
determining a start of combustion of a fuel injected for each of the cycles;
calculating the mean start of combustion angle “θ_{mean start}” from the start of combustion “θ” values recorded for the set of n cycles;
determining an end of combustion of a fuel injected for each of the cycles;
calculating the mean end of combustion angle “θ_{mean end}” from the end of combustion “θ” values recorded for the set of n cycles;
calculating the combustion delay between the start of combustion time found from “θ_{mean start}” and the end of combustion time found from “θ_{mean end}”;
deducing the ethanol content from the combustion delay, on the basis of a determined model or of a look-up list providing the relationship between the combustion delays and the ethanol contents.

According to other advantageous and nonlimiting features of this method:
said ethanol content is stored in memory until a different ethanol content is learnt;
for various successive angles that the crankshaft of the engine adopts within each cycle, a variable
“C_{θ}” is evaluated at an angle “θ” of the crankshaft in the combustion chamber of the cylinder, “C_{θ}” being based on the angular derivative

$$\frac{d Q}{d θ}$$

of the quantity of energy “Q” at an angle “θ”, and the angular value CA10, which is equal to the angle at which

$$Q = \int \frac{d Q}{d θ}$$

reaches 10% of its maximum amplitude AM is calculated, this value CA10 being recorded as being the start of combustion angular value for the cycle in question;
for various successive angles that the crankshaft of the engine adopts within each cycle, a variable “C_{θ}” is evaluated at an angle “θ” of the crankshaft in the combustion chamber of the cylinder, “C_{θ}” being based on the angular derivative

$$\frac{d Q}{d θ}$$

of the quantity of energy “Q” at an angle “θ”, and the angular value CA90, which is equal to the angle at which

$$Q = \int \frac{d Q}{d θ}$$

reaches 90% of its maximum amplitude AM is calculated, this value CA90 being recorded as being the end of combustion angular value for the cycle in question;
said maximum amplitude AM is measured by establishing the difference between:
   the maximum and minimum values of Q; or
   the maximum value of Q and zero; or
   zero or the minimum value of Q, and the angle at the end of integration of Q if this corresponds to the maximum value of Q;
said variable “C_{θ}” is calculated from the following expression:

$$- \frac{d Q}{d θ} = \frac{V}{γ - 1} ( \frac{p}{T} \frac{d T}{d θ} ) + p \frac{d V}{d θ}$$

“p” being the pressure in the cylinder at the angle “θ”,
a “V” being the volume of the combustion chamber at the angle “θ”,
“T” being the temperature in the combustion chamber at the angle “θ”,
“γ” being the ratio of specific heat capacities “cₚ/cᵥ”.

Other features and advantages of the invention will become apparent from reading the detailed description which will follow of a preferred embodiment of the invention.

This description will be given with reference to the attached drawings in which:

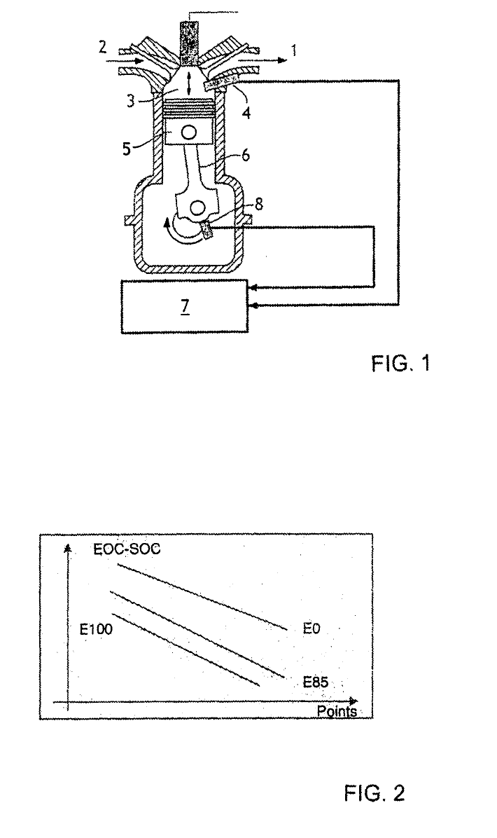

FIG. 1 schematically depicts a cross section through a cylinder of a combustion engine in operation;

FIG. 2 is a schematic depiction of the change in the variable “EOC-SOC” as a function of the number of points, which makes it possible to determine the ethanol content in a given fuel.

FIG. 1 illustrates a device according to the invention. It relates to an internal combustion gasoline engine comprising a moving piston 5, a connecting rod 6, a combustion chamber 3, a gas inlet device 2, a gas exhaust device 1, a measurement means capable of measuring various angles “θ” of rotation of the crankshaft 8, a pressure sensor 4 positioned in the combustion chamber 3, and processing means 7 capable of receiving the signals from said pressure sensor 4 and from the measurement means 8, of synchronizing these signals in order to be able to correlate them, and of processing these signals in order to implement a method according to the invention.

When the engine is running, an estimate of the ethanol content is performed according to the invention. This is advantageously done under operating conditions set in advance during engine development.

The ethanol content of the fuel is estimated, as will be seen later on, by taking account of the rate of combustion, namely the time that elapses between the time that combustion starts and the time that it ends.

The activation conditions useful in implementing the present invention are the operating points, preferably at high engine load, for estimating the start and end of combustion most easily. Nonetheless, it would also appear to be advantageous to choose phases such as engine start (ensuring suitable external conditions), which is a phase during which the type of fuel is of key importance. All of these activation conditions may take the form of one engine operating point or of several. A learning phase may also be demanded each time the tank is refilled. The activation conditions are then awaited in order to start the learning phase.

When the activation conditions have been recognized (that is to say when the engine is under the action of the driver on the or one of the identified operating points), the start and end of combustion is detected over n thermodynamic cycles (n may be equal to 1).

It is therefore first and foremost essential to determine the start of combustion (also termed “SOC”) and end of combustion (or “EOC”) instances.

One possible way of determining the SOC and the EOC is based on calculating and analyzing a variable C_{θ} that provides an image of the combustion.

The methodology set out hereinafter is also set out in the aforementioned application 06/07041.

Using the value of the pressure p_{θ} obtaining in the combustion chamber, and the value of the angular position θ of the crankshaft, which values have been measured during a first step, a variable C_{θ} is permanently calculated during a later step.

C_{θ} is a function of an angular change in the quantity of energy “Q” released by the combustion in the combustion chamber 3 at an angle θ.

This angular change in the quantity of energy is advantageously the derivative

$$\frac{d Q}{d θ} .$$

This value C_{θ} is permanently calculated on the basis of the release of energy in the cylinder, for an angle δ changing between −180° and +180° (i.e. in a half-cycle), or in a smaller interval.

According to a first option, C_{θ} is calculated from:

$$C = \frac{1}{F} \frac{d Q}{d θ} \qquad ( 1 )$$

where “Q” is the energy of the cylinder,
“θ” is the crank angle, and
“F” is a function of “θ” such that “F” is at a maximum at bottom dead center (also denoted BDC), at a minimum at top dead center (also denoted TDC), monotonous between the two, and always strictly positive.

A “triangle” function has such properties, and may be defined as follows:

F=F₀+K|θ| (2)

where “K” and “F₀” are constants.

In practice, the calculation of the release of energy

$$\frac{d Q}{d θ}$$

provides information containing noise.

The effect of such a function “F” is to attenuate the energy release signal in the regions where it is affected by the most noise (on the BDC side), by comparison with the regions in which it is liable to contain the information of interest (on the TDC side).

This operation therefore increases the signal-to-noise ratio and reduces the pressure filtering constraints that need to be applied.

According to a second option, C_{θ} is calculated from the following simplified expression for the release of energy in the cylinder per unit volume:

$$C = \frac{1}{V} \frac{d Q}{d θ} \qquad ( 3 )$$

where V represents the volume of the combustion chamber 3 for an angle θ.

Because the volume varies cyclically with θ, multiplying by

$$\frac{1}{V}$$

has the advantage of reducing the amplitude of the noise on the release of energy at the places where it is typically the highest (near BDC). This calculation also amplifies the signal in the places where it is of use for detecting the SOC and the EOC (typically found near TDC).

This operation therefore increases the signal-to-noise ratio and reduces the pressure filtering constraints that have to be applied.

In practice, calculating

$$\frac{d Q}{d θ}$$

may prove complicated and may therefore entail computation power in order to be performed as such in an onboard electronic engine management system. However, dividing by the volume is of benefit because it allows the simplifications set out hereinbelow to be applied.

We start out from the expression, well known to those skilled in the art, of the apparent release of energy

$$\frac{d Q}{d θ}$$

of the gas present in the chamber:

$$\frac{d Q}{d θ} = \frac{1}{γ - 1} V ( \frac{p}{m} \frac{d m}{d θ} + \frac{p}{T} \frac{d T}{d θ} - \frac{p}{V} \frac{d V}{d θ} ) + \frac{γ}{γ - 1} p \frac{d V}{d θ} \qquad ( 4 )$$

where:
Q is the quantity of energy present in the gases in the combustion chamber 3;
θ is the crank angle;
γ is the ratio of specific heat capacities (cₚ and cᵥ);
V is the volume of the combustion chamber at θ;
p is the pressure in the combustion chamber at θ;
m is the mass of gases trapped in the cylinder at θ;
T is the temperature in the combustion chamber θ.

It should be noted that expression (4) could be rewritten, using time t in place of θ.

However, it is preferable to use the angle θ, because the system then becomes independent of the rotational speed of the engine.

It should also be noted that expression (4) taken as a starting point contains the angular derivative of the temperature rather than the derivative of the pressure.

That makes it possible to minimize the noise, because the derivative of the measured pressure p introduces a great deal of noise into the result.

Advantageously, only the pressure p and the angle θ are values measured by the device according to FIG. 1.

The other variables in expression (4) are either neglected or estimated, as we shall see.

Regarding the mass m, the assumption is made to consider this constant (the injected mass is therefore neglected). The expression for C then becomes:

$${C}_{θ} = \frac{1}{V} \frac{d Q}{d θ} = \frac{1}{γ - 1} ( \frac{p}{T} \frac{d T}{d θ} ) + \frac{p}{V} \frac{d V}{d θ} \qquad ( 5 )$$

The ratio of specific heat capacities γ is considered to be constant before combustion and in the first few moments of combustion. It may, for example, be estimated as being equal to 1.4.

For a given engine geometry, there is an unchanging relationship between the volume V and the angle θ. To estimate the volume V and its derivative

$$\frac{d V}{d θ} ,$$

a table of values giving the volume V for different values of θ is used.

The temperature T and its derivative

$$\frac{d T}{d θ}$$

are calculated on the basis of said volume V and pressure p.

According to a first way of calculating T, it is assumed that the gas is a perfect gas. It can therefore be written that, at any instant:

$$\frac{pV}{T} = mr = constant \qquad ( 6 )$$

Thus, at two different moments or two different angles:

$$\frac{{p}_{θ} {V}_{θ}}{{T}_{θ}} = \frac{{p}_{0} {V}_{0}}{{T}_{0}} \qquad ( 7 )$$

From this we can deduce:

$${T}_{θ} = {T}_{0} \frac{{p}_{θ} {V}_{θ}}{{p}_{0} {V}_{0}} \qquad ( 8 )$$

where:
T₀, p₀, V₀ are the initial temperature, pressure and volume (i.e. the values upon closure of the inlet valve).
T_{θ}, p_{θ}, V_{θ} are the temperature, pressure and volume at the angle θ.

The temperature is therefore estimated for each value of the angle θ, from initial pressure, temperature and volume conditions at the start of the cycle, preferably just after the inlet valve closes.

The initial temperature can be considered to be constant and equal to 300K, or alternatively, it can be made to depend on the admitted-air temperature, if this is being measured.

This method may be preferred because it entails very few calculations, given that

$$\frac{{T}_{0}}{{p}_{0} {V}_{0}}$$

is calculated just once per cycle and this result need merely be multiplied by p_{θ}V_{θ} for each new value of the angle θ.

According to a second way of calculating T, it is again assumed that the gas is a perfect gas (pV=mrT, where mr is constant), but this time it is the following that is deduced from this:

$$\frac{1}{T} \frac{d T}{d θ} = \frac{1}{p} \frac{d p}{d θ} + \frac{1}{V} \frac{d V}{d θ} \qquad ( 9 )$$

This expression could be reintroduced into the expression for C_{θ}, but that would cause the derivative of pressure

$$\frac{d p}{d θ}$$

to reappear, and this derivative is a source of noise in the calculation.

A different way of expressing

$$\frac{1}{T} \frac{d T}{d θ}$$

will therefore be preferable.

To do that, expression (9) is discretized and the values adopted by V, p, T are considered at the angles θ and θ−Δθ, where Δθ is constant and represents the step size of the calculation. Expression (9) then becomes:

$$\frac{1}{{T}_{θ}} \frac{{T}_{θ} - {T}_{θ - Δ θ}}{Δ θ} = \frac{1}{{p}_{θ}} \frac{{p}_{θ} - {p}_{θ - Δ θ}}{Δ θ} + \frac{1}{{V}_{θ}} \frac{{V}_{θ} - {V}_{θ - Δ θ}}{Δ θ} \qquad ( 10 )$$

The following expression for the temperature T at the angle θ is then deduced, as a function of the temperature at the angle θ−Δθ:

$${T}_{θ} = {T}_{θ - Δ θ} ( \frac{1}{\frac{{p}_{θ - Δ θ}}{{p}_{θ}} + \frac{{V}_{θ - Δ θ}}{Δ θ} - 1} ) \qquad ( 11 )$$

The temperature is therefore estimated recursively for each value of the angle θ, from an initial temperature corresponding to the temperature in the cylinder at the moment the inlet valve closes. It may be considered to be constant and equal to 300K, or alternatively may be dependent on an admitted-air temperature measured by appropriate means.

To finish off, the expression of C_{θ} is also discretized, and the value of C_{θ} for each value of θ is given by the formula:

$${C}_{θ} = \frac{1}{γ - 1} ( \frac{{p}_{θ}}{{T}_{θ}} \frac{{T}_{θ} - {T}_{θ - Δ θ}}{Δ θ} ) + \frac{{p}_{θ}}{{V}_{θ}} \frac{{V}_{θ} - {V}_{θ - Δ θ}}{Δ θ} \qquad ( 12 )$$

Furthermore, expression (9), (11) or (12) can be simplified still further, in terms of computation, by simplifying the expression for V (or V_{θ}).

Specifically, as stated hereinabove, the law relating to the volume V, which is unchanging for a given engine geometry, may be determined by a table of values, associating the volume value with each value of θ.

In an onboard system, it is nonetheless advantageous to reduce the size of the tables of values because these use up memory resources. The simplification set out hereinafter will at the very least make it possible to reduce the size of the tables of values held in memory, and at most, make it possible to dispense with these.

Rather than using the true volume in the calculations set out hereinabove, use is made in its place of a parabolic law Vₚ given by the expression: Vₚ=V₀+Kₚθ² where V₀ is constant and is the volume at TDC (dead volume),

Kₚ is a coefficient that needs to be adjusted so that the curve of Vₚ is very close to the curve of V near TDC.

Vₚ may thus be substituted for V (or V_{θ}) throughout the aforementioned calculations: for calculating the temperatures T_{θ} and T_{θ-Δθ} and in the term

$$\frac{{p}_{θ}}{{V}_{θ}} \frac{{V}_{θ} - {V}_{θ - Δ θ}}{Δ θ} .$$

Computation time is therefore saved and resources are economized.

According to the invention, the end of combustion “EOC” calculation can be performed through calculating the “CA90” rather than by estimating the end of combustion using the method set out above.

“CA90” is the crank angle, for a determined cycle, at which 90% of the charge present in the combustion chamber has been burnt.

It corresponds to the angle at which

$$Q = \int \frac{d Q}{d θ}$$

(where Q and θ are as explained above) reaches 90% of its maximum amplitude AM.

This maximum amplitude AM may be measured by calculating the difference between:
the maximum and minimum values of Q; or
the maximum value of Q and zero; or
zero or the minimum value of Q, and the angle at the end of integration of Q if this corresponds to the maximum value of Q.

Once the SOC and EOC have been detected, the corresponding crank angles are recorded.

The values of these n angles are stored in memory and their means are determined.

The difference between these two values EOC-SOC provides an image of the rate of combustion.

The calculated value EOC-SOC is injected into a map of the type in the example of attached FIG. 2.

The output from this map provides the ethanol content in the fuel that is in the process of being consumed.

The value from the aforementioned map is stored in memory in appropriate means until a different ethanol content is learnt.

The engine management system calibrations are then adapted to this ethanol content value (ignition advance maps for example) to guarantee optimal engine running.

The rate of combustion EOC-SOC can be estimated by calculating the CA10, rather than by estimating the start of combustion using the method set out above.

The CA10 is the angle at which 10% of the charge present in the combustion chamber has been burnt. It corresponds to the angle at which

$$Q = \int \frac{d Q}{d θ}$$

reaches 10% of its maximum amplitude AM.

This maximum amplitude AM can be measured by calculating the difference between:
the maximum and minimum values of Q;
   or
the maximum value of Q and zero;
   or
zero or the minimum value of Q, and the angle at the end of integration of Q if this corresponds to the maximum value of Q.

1-6. (canceled)

7. A method for estimating ethanol content of a fuel injected into an internal combustion engine, over a number n of cycles (n being an integer greater than or equal to 1) of displacement of a piston in a cylinder, comprising:
determining a start of combustion of a fuel injected for each of the cycles;
calculating the mean start of a combustion angle from the start of combustion values recorded for the n cycles;
determining an end of combustion of a fuel injected for each of the n cycles;
calculating the mean end of combustion angle from the end of combustion values recorded for the n cycles;
calculating a combustion delay between the calculated start of combustion time and the calculated end of combustion time; and
deducing ethanol content from the combustion delay, on the basis of a determined model or a look-up list providing the relationship between the combustion delays and the ethanol contents.

8. The method as claimed in claim 7, wherein the ethanol content is stored in memory until a different ethanol content is learned.

9. The method as claimed in claim 7, wherein, for plural successive angles that a crankshaft of the engine adopts within each cycle, a variable C_{θ} is evaluated at an angle θ of the crankshaft in the combustion chamber of the cylinder, the variable C_{θ} being based on angular derivative
$$\frac{d Q}{d θ}$$
of the quantity of energy Q at an angle θ, and an angular value, which is equal to the angle at which
$$Q = \int \frac{d Q}{d θ}$$
reaches 10% of its maximum amplitude is calculated, the angular value being recorded as a start of combustion angular value for the cycle in question.

10. The method as claimed in claim 7, wherein, for plural successive angles that the crankshaft of the engine adopts within each cycle, a variable C_{θ} is evaluated at an angle θ of the crankshaft in the combustion chamber of the cylinder, C_{θ} being based on the angular derivative
$$\frac{d Q}{d θ}$$
of the quantity of energy Q at an angle θ, and an angular value, which is equal to the angle at which
$$Q = \int \frac{d Q}{d θ}$$
reaches 90% of its maximum amplitude is calculated, the angular value being recorded as being the end of combustion angular value for the cycle in question.

11. The method as claimed in claim 9, wherein the maximum amplitude is measured by establishing the difference between:
the maximum and minimum values of Q;
or
the maximum value of Q and zero;
or
zero or the minimum value of Q, and the angle at the end of integration of Q if this corresponds to the maximum value of Q.

12. The method as claimed in claim 9, wherein the variable C_{θ} is calculated from the following expression:
$$\frac{d Q}{d θ} = \frac{V}{γ - 1} \frac{p}{T} \frac{d T}{d θ} + p \frac{d V}{d θ}$$
p being the pressure in the cylinder at the angle θ,
V being the volume of the combustion chamber at the angle θ,
T being the temperature in the combustion chamber at the angle θ,
γ being the ratio of specific heat capacities cₚ/cᵥ.
